(19) United States
(12) Patent Application Publication
(10) Pub. No.: US 2025/0295485 A1
(43) Pub. Date: Sep. 25, 2025

(54) EXPANDABLE TISSUE ANCHOR

(71) Applicant: Teleflex Life Sciences LLC, Wilmington, DE (US)

(72) Inventors: Alexander Charles Gordon, San Carlos, CA (US); Curtis Yarra, Oakland, CA (US); Erik Noel, San Ramon, CA (US); Ernest Cabreza, Dublin, CA (US)

(73) Assignee: Teleflex Life Sciences LLC, Wilmington, DE (US)

(21) Appl. No.: 19/085,988

(22) Filed: Mar. 20, 2025

Related U.S. Application Data
(60) Provisional application No. 63/568,793, filed on Mar. 22, 2024.

(51) Int. Cl. A61F 2/04

(52) U.S. Cl. CPC A61F 2/04; A61F 2002/047

(57) ABSTRACT

A prostatic implant configured to compress an enlarged prostate gland includes a distal anchor member and an expandable, proximal anchor member connected by a middle portion. The expandable, proximal anchor member is configured to expand upon being released from a tubular delivery device, such that the anchoring footprint of the expandable anchor member on the urethral side of a prostate gland is large relative to anchors having fixed dimensions. The expandable anchor member includes expandable portions configured to expand outwardly or laterally away from the body of the expandable anchor member, forming curved portions or wings. After anchoring the distal anchor member to the outer surface of the prostatic capsule, tension may be applied to the middle portion extended through the prostate gland. Attachment of the expandable anchor member to the middle portion at the urethral side of the gland locks the tension in place, retracting the enlarged prostatic tissue.

### CROSS-REFERENCE TO RELATED APPLICATIONS

This application claims the benefit of priority to U.S. Provisional Appln. Ser. No. 63/568,793, filed Mar. 22, 2024, which is incorporated herein by reference in its entirety.

### TECHNICAL FIELD

The subject matter of this patent document relates to the field of medical devices. More particularly, but not by way of limitation, the subject matter relates to medical devices, systems, and methods for relieving pressure on a prostatic urethra by compressing at least a portion of a prostate gland.

### BACKGROUND

Benign Prostatic Hyperplasia (BPH) is one of the most common medical conditions that affect men, especially elderly men. It has been reported that, in the United States, more than half of all men have histopathologic evidence of BPH by age 60 and, by age 85, approximately 9 out of 10 men suffer from the condition. Moreover, the incidence and prevalence of BPH are expected to increase as the average age of the population in developed countries increases. Despite extensive efforts in both the medical device and pharmacotherapeutic fields, current treatments remain only partially effective and are burdened with significant side effects. Thus, there remains a need for the development of new devices, systems, and methods for treating BPH as well as other conditions in which one tissue or anatomical structure impinges upon or compresses another tissue or anatomical structure.

### SUMMARY

Embodiments disclosed herein include devices, systems, and methods for compressing at least a portion of a prostate gland to alleviate pressure on a prostatic urethra. Embodiments include a prostatic implant configured to anchor simultaneously to the outer prostatic capsule, and also a urethral side, of the lobe of an enlarged prostate, such as a median or lateral lobe. The implant may include a distal anchor portion configured to anchor on the outside of the prostatic capsule. An elongate middle portion. e.g., a suture, may connect the distal anchor portion to an expandable, proximal anchor portion or urethral endpiece configured to anchor to a urethral side of the lobc. Once the distal anchor portion is implanted, the elongate middle portion may be tensioned and the expandable, proximal anchor portion subsequently attached thereto. Attachment of the proximal anchor portion may lock the tensioned middle portion in place, compressing the prostatic tissue between the distal and proximal anchors and relieving constriction of the prostatic urethra.

The expandable anchor portion of the implant may be configured to expand and change shape depending on its position within or outside a delivery device, which may include an elongate tubular member. For instance, the expandable anchor portion may assume a smaller, compressed configuration when constrained within a delivery device, and a larger, expanded configuration when released from the delivery device. The expandable anchor portion may thus be biased toward an expanded configuration, which may be attained upon its release from a delivery device at a targeted tissue site, such as at or near the urethral side of at least a portion of a prostate gland. Relative to preexisting prostatic implants having fixed dimensions, the anchoring footprint of the expandable anchor portion may be larger, which may increase tissue retraction.

These and other examples and objects of the present devices, systems, and related methods will be set forth in the following Detailed Description. This Overview is intended to provide non-limiting examples of the present subject matter. The Detailed Description below is included to provide further information about the present devices, systems, and related methods. Neither is not intended to provide an exclusive or exhaustive explanation of the present devices, systems, and methods because this disclosure is written for those of ordinary skill in the art.

### BRIEF DESCRIPTION OF THE DRAWINGS

In the drawings, like numerals can be used to describe similar features and components throughout the several views. The drawings illustrate generally, by way of example but not by way of limitation, various embodiments discussed in the present patent document.

FIG. 1 illustrates a cross-sectional view of the anatomy surrounding a prostate in a human subject.

FIG. 2 illustrates an enlarged cross-sectional view of the anatomy surrounding a prostate.

FIG. 3 illustrates an implant in accordance with embodiments of the present disclosure.

FIG. 4A illustrates the expandable anchor member of the implant shown in FIG. 3 in a constrained delivery configuration.

FIG. 4B illustrates the expandable anchor member of the implant shown in FIG. 3 in an unconstrained anchoring configuration.

FIG. 5 illustrates a side view of a delivery device for an implant in accordance with embodiments of the present disclosure.

FIG. 6 illustrates a depiction of a step performed during an implantation procedure in accordance with embodiments of the present disclosure.

FIG. 7A illustrates another expandable anchor member of an implant in a constrained configuration in accordance with embodiments of the present disclosure.

FIG. 7B illustrates the expandable anchor member shown in FIG. 7A in an unconstrained configuration.

FIG. 8A illustrates another expandable anchor member of an implant in a constrained configuration in accordance with embodiments of the present disclosure.

FIG. 8B illustrates the expandable anchor member shown in FIG. 8A in an unconstrained configuration.

FIG. 9 illustrates another expandable anchor member of an implant in an unconstrained configuration in accordance with embodiments of the present disclosure.

FIG. 10 illustrates a flow diagram of a method of delivering an implant to a prostate in accordance with embodiments of the present disclosure.

The drawing figures are not necessarily to scale. Certain features and components may be shown exaggerated in scale or in schematic form and some details may not be shown in the interest of clarity and conciseness.

### DETAILED DESCRIPTION

The present devices and associated methods provide clinicians with a means to treat an enlarged prostate, which may be a symptom of benign prostatic hyperplasia, to alleviate its impingement on the adjacent prostatic urethra. Implants disclosed herein can be placed using a method for compressing a prostate gland or portion thereof according to the following description.

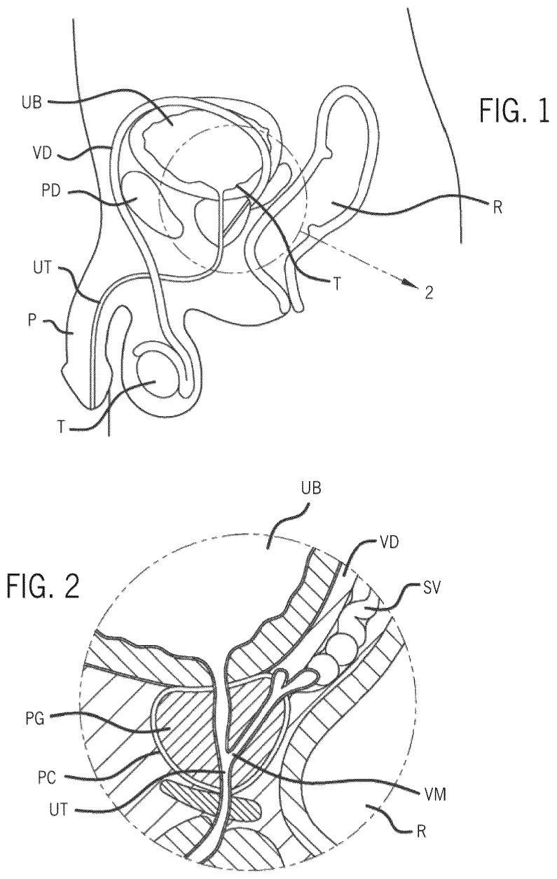

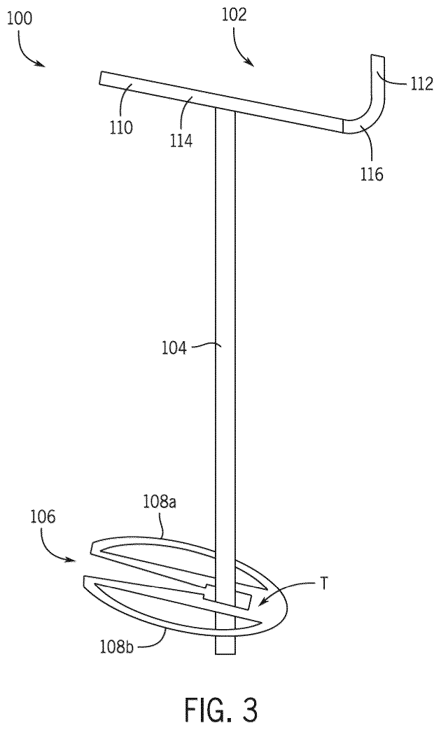

FIGS. 1 and 2 illustrate various features of the urological anatomy of a human subject. The prostate gland PG is a walnut-sized muscular gland located adjacent the urinary bladder UB. The urethra UT runs through the prostate gland PG. The prostate gland PG secretes fluid that protects and nourishes sperm. The prostate also contracts during sperm ejaculation to expel semen and provide a valve to keep urine out of the semen. A firm prostatic capsule PC surrounds the prostate gland PG.

The urinary bladder UB holds urine. The vas differentia VD define ducts through which semen is carried, and the seminal vesicles SV secrete seminal fluid. The rectum R is the end segment of the large intestine through which waste is dispelled. The urethra UT carries both urine and semen out of the body. Thus, the urethra is connected to the urinary bladder UB and provides a passageway to the vas differentia VD and seminal vesicles SV.

The trigone T is a smooth triangular end of the bladder. It is sensitive to expansion and signals the brain when the urinary bladder UB is full. The verumontanum VM is a crest in the wall of the urethra UT where the seminal ducts enter. The prostatic urethra is the section of the urethra UT that extends through the prostate.

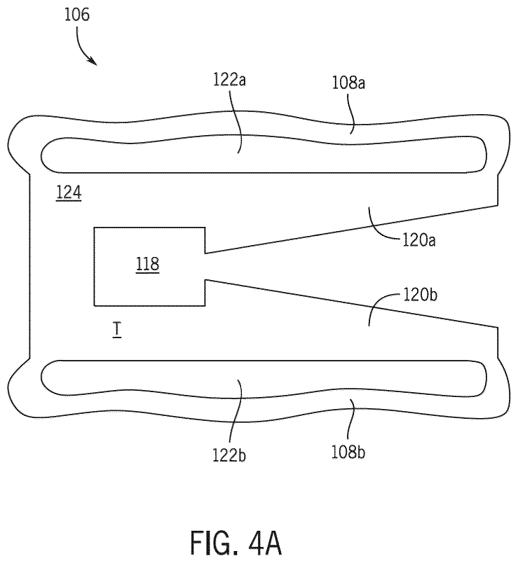

FIG. 3 illustrates an embodiment of an implant 100 used to compress at least a portion of a prostate gland in a subject. The example shown includes a distal end portion or anchor member 102, a tension element or middle portion 104, and a proximal end portion, urethral endpiece or expandable anchor member 106. The distal anchor member 102 may be configured to engage with and anchor to the outer surface of a prostatic capsule of a lobe of a prostate gland, and the proximal, expandable anchor member 106 may be configured to engage with and anchor to the urethral side of the lobe, with the middle portion 104 configured to pass through the lobe, connecting the distal and proximal anchor members. The middle portion 104 may comprise a suture or suture portion that can be tightened or tensioned upon implantation. The implant 100 is shown in an expanded, unconstrained anchoring configuration, with expandable members, wings or curved portions 108a, 108b of the expandable anchor member 106 extended generally laterally or outwardly relative to the longitudinal axis of the middle portion 104. The unconstrained anchoring configuration may be the native, relaxed state of the device configured for anchoring to a lobe of a prostate gland. The top surface T of the implant 100 is visible in FIG. 3. The top surface and bottom surface may be identical and interchangeable.

The configuration of one or more components of the implant 100 may vary. Embodiments of the distal anchor member 102, for example, may include a variety of modified, additional, or substitute components, compositions, features, and/or configurations, such as those of distal anchor 70 described in U.S. Pat. No. 9,034,001, the entire contents of which are incorporated by reference herein. As shown in FIG. 3, one non-limiting example of the distal anchor member 102 may comprise a tubular portion 110 and a tail portion 112 connected by a mid-section 114 that provides a structural transition from the tubular portion 110 and the tail portion 112. A connector section 116 may extend from the mid-section 114. The connector section 116 may include a bend that creates an orthogonally or obliquely oriented configuration, which may act as a barb or deflected strut to cause rotation of the distal anchor member 102 relative to the middle portion 104 after deployment from an elongate delivery device (creating a “flipping tail”) and produce the relative unconstrained (orthogonally oriented) angle assumed between the tail portion 112 and tubular portion 110 of the distal anchor member 102. The distal anchor member 102 may be configured to be constrained to a generally straight configuration aligned with a longitudinal axis of the elongate delivery device when positioned therein, prior to deployment. The distal anchor member 102 may comprise an electro-polished Nitinol (nickel titanium alloy SE508, 50.8% nickel) tube in some examples.

Additional examples of the distal anchor member 102 may not be tubular in whole or in part and/or may comprise additional or alternative materials, e.g., one or more metals, alloys, and/or polymers. Embodiments of the distal anchor member 102 may comprise various components that couple with the middle portion 104 and/or one or more components of an elongate delivery device, which may comprise an elongate tubular member and/or delivery needle, via a variety of coupling mechanisms. The configuration of the distal anchor member 102 and its attachment to the implant 100 and/or prostatic lobe may thus vary.

Embodiments of the tensionable middle portion 104 may include a suture comprising a polyester or monofilament, e.g., Poly Ethylene Terephthalate (PET). Non-limiting examples may include a suture comprised of a round monofilament extrusion/pultrusion, which may be composed of a grade 8816 polyethylene terephthalate. In embodiments featuring a suture, the base material may be annealed at approximately 100 degrees Celsius for approximately 130 minutes in a straight condition. In one embodiment, the middle portion 104 may comprise a PET suture having a diameter of 0.015 inches and a tensile strength greater than or equal to about 12.7 pounds. Other suitable materials, dimensions, and manufacturing parameters may be used.

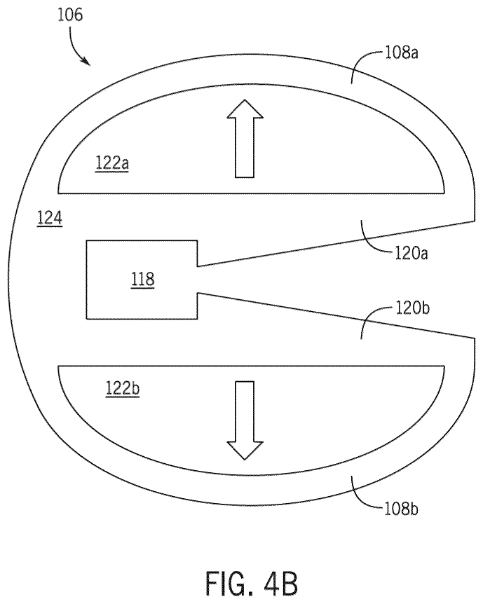

Additional features and configurations of the expandable anchor member 106 are shown in FIGS. 4A and 4B FIG. 4A is a plan view of the expandable anchor member 106 in a compressed or constrained delivery configuration, with the expandable portions 108a, 108b compressed toward a seating region 118 configured to receive the middle portion 104. The expandable anchor member 106 may assume the constrained delivery configuration to fit within a delivery device, for example when positioned within an inner lumen of an elongate tubular member configured to navigate the urethra with the implant 100 housed therein. At a target site adjacent to a lobe of a prostate gland, the expandable anchor member 106 may exit the delivery member through a sidewall opening in the shaft of the delivery member, allowing the expandable anchor member 106 to expand.

The expandable anchor member 106 may feature two prongs or prong-like portions 120a, 120b that, together with the expandable portions 108a, 108b, may define two expansion gaps 122a, 122b. A backend or body portion 124 of the expandable anchor member 106 may comprise the prong portions 120a, 120b together with the material surrounding and defining the seating region 118. Some embodiments of the body portion 124 may comprise all but the expandable portions 108a, 108b of the expandable anchor member 106.

The middle portion 104 of the implant 100 may be positioned orthogonally or approximately perpendicularly to the depicted top (and bottom) of the expandable anchor member 106, such that the middle portion 104 extends transversely through the seating region 118 (into and out of the page) upon coupling of the two components, which may occur after the implant 100 reaches a target site at the urethral side of a lobe of a prostate gland. The prong portions 120a, 120b may grip and in some examples deform the middle portion 104. Accordingly, the expandable anchor member 106 may be secured to the suture with a compressive force, while also expanding outwardly to provide a larger surface area and footprint on the surface of the urethral side of a targeted lobe.

The size and shape of the seating region 118 of the expandable anchor member 106 may vary. In the example shown, the seating region 118 is approximately rectangular, but it may also be approximately circular, oblong, square, triangular, or any other suitable geometry. The seating region 118 may also comprise a narrow slit through a portion of the body portion 124. The slit may be connected to the space between the two prong portions 120a, 120b. The seating region 118 may have a cross-sectional shape that matches or approximately matches the cross-sectional size and shape of the middle portion 104, for instance such that the cross-sectional shape of the seating region 118 may be substantially circular when coupled with a middle portion 104 having a substantially circular cross-section. The cross-sectional diameter of the seating region 118 may be smaller than the cross-sectional diameter of the middle portion 104, such that when the middle portion 104 is pushed into the seating region 118, the middle portion 104 deforms where it contacts the seating region 118, locking the middle portion into the expandable anchor member.

The prong portions 120a, 120b may be relatively stiff and therefore subject to minimal to no deflection. The prong portions 120a, 120b may guide the middle portion 104 into the seating region 118, which then bites into the middle portion 104 to securely lock the middle portion 104 to the expandable anchor member 106.

The seating region 118 is shown closer to one end of the expandable anchor member 106 than the other. Additional embodiments may feature a seating region positioned at approximately the center or midpoint of the expandable anchor member 106.

FIG. 4B illustrates a plan view of the expandable anchor member 106 in an expanded, unconstrained anchoring configuration. As indicated by the arrows, the expandable portions 108a, 108b have expanded outwardly, away from the body portion 124, such that the expansion gaps 122a, 122b are larger relative to the constrained delivery configuration shown in FIG. 4A. The unconstrained anchoring configuration may be attained upon release of the expandable anchor member 106 from a delivery device having an inner diameter that is smaller than a width of the expandable anchor member 106. No longer bound within the delivery device, the expandable portions 108a, 108b may spring or otherwise expand laterally or outwardly from the longitudinal axis of the delivery device, enlarging the surface area and footprint of the expandable anchor member 106 such that it anchors to the urethral side of the lobe in a manner that reduces the likelihood of cutting or tearing therethrough while also increasing tissue retraction, which may encompass a greater volume of retracted tissue and/or greater compression exerted on the retracted tissue.

The configuration of the expandable portions 108a, 108b may vary. The example shown includes two wing-like arcuate members after expansion. Additional examples may feature angled expandable portions resembling a variety of shapes together with each prong portion after expansion. For example, each expandable portion may comprise two straight portions converging away from the body portion 124 such that after expansion, the two straight portions and adjacent prong resemble a triangle. Additional expandable portions may feature more than two straight portions, which may or may not connect. For instance, each expandable portion may comprise two or more straight portions that extend separately away from the body portion 124 in the unconstrained configuration such that after expansion, each expandable portion resembles an eyelash shape.

The expandable portions may be formed integrally with or attached to the body portion 124 in different ways. Embodiments may feature expandable portions attached to the body portion 124 via a hinge-like mechanism, for instance. Additional examples, such as the one shown in FIGS. 4A and 4B, may feature expandable portions configured to expand away from the body portion 124 due to the biased state and/or material composition of the expandable portions 108a, 108b.

The material composition of the expandable portions 108a, 108b may be substantially flexible or elastic to enable their compression within a delivery device and subsequent expansion when released therefrom. Expansion of the expandable portions 108a, 108b may involve or resemble an outward spring action. Non-limiting example materials constituting the expandable portions 108a, 108b may include a flexible stainless steel, a shape memory Nitinol, one or more metals or alloys, and/or a polymer of suitable elasticity.

Embodiments of the expandable anchor member 106 may include a stainless steel body and expandable portions 108a, 108b comprising Nitinol or spring-temper steel. In additional embodiments, the body and expandable members of the expandable anchor portion may comprise the same material composition, e.g., Nitinol or spring-temper steel. Accordingly, the entire expandable anchor member 106, including the expandable portions 108a, 108b, may be formed from a single material composition, or the expandable portions 108a, 108b may be formed from a material composition that is different than one or more additional material compositions constituting the expandable anchor member 106.

The implant 100 may be delivered to a targeted lobe of a prostate gland using a delivery system that further includes a delivery device comprising a tubular elongate member and at least one hollow delivery needle configured to be advanced therethrough. The needle may have a sharp distal tip configured to pierce the prostate gland, including the outer capsule, along with an inner lumen configured to house the distal anchor member 102 and middle portion 104 therein, ready for deployment.

Examples of the delivery device may generally include a handle assembly supporting an elongate portion comprising a tubular elongate member in the form of or comprising a shaft. The elongate member may be substantially rigid or flexible and defines a low profile suited to navigate body anatomy to reach an interventional site. Examples of the elongate member may define two inner lumens, with one lumen configured to accommodate extension of the distal anchor member 102 and middle portion 104 therethrough, and a second inner lumen configured to accommodate the expandable anchor member 106 in its constrained configuration. Substructure may be provided to maintain a longitudinal profile of the elongate member so that the interventional procedure can progress as intended. Embodiments of the delivery device may also include an endoscope, providing the ability to view the interventional procedure. Using the disclosed systems, insertion of one or more implants in a prostate gland may be performed in an outpatient setting.

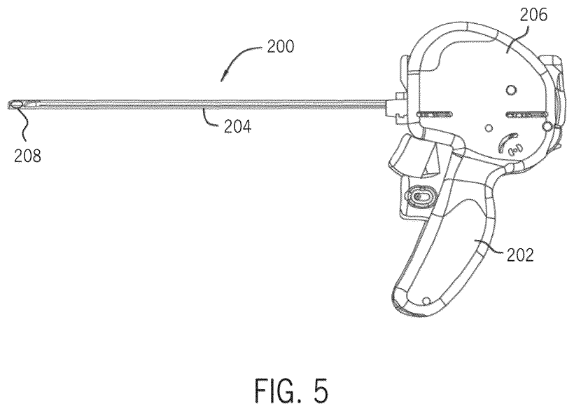

FIG. 5 illustrates one example of a delivery device 200 having structure configured to gain access to an interventional site and deploy a prostatic implant, such as implant 100. As shown, the delivery device 200 may include a handle assembly 202 connected to a tubular elongate member 204, which may surround an elongate delivery needle defining an inner lumen and arranged coaxially to the elongate member 204. The elongate member 204 may be sized to fit within a cystoscopic sheath for patient tolerance during a procedure in which the subject is awake rather than under general anesthesia. Non-limiting examples of the sheath may be 19 F or 20 F.

The delivery device 200 may further include a number of subassemblies configured to deliver and employ an implant at a target site. A handle case assembly 206, including handle parts that form part of the handle assembly 202. The handle assembly 202 is sized and shaped to fit comfortably within an operator's hand and can be formed from conventional materials. Windows can be formed in the handle case assembly 206 to provide access to internal mechanisms of the device so that a manual override is available to the operator in the event the interventional procedure needs to be abandoned.

The elongate member 204 defines at least one inner lumen sized and configured to accommodate longitudinal insertion of at least the hollow delivery needle and prostatic implant therethrough, with the distal anchor member of the implant nestled within the delivery needle, and the expandable anchor member compressed within the elongate member 204 in the constrained delivery configuration. In some examples, the distal anchor member and middle portion may be enclosed within a first lumen of the elongate member 204, and the expandable anchor member may be enclosed within a second lumen of the elongate member 204. The elongate member 204 may have a shape and/or flexibility configuring it to navigate through a urethra without kinking or puncturing the urethral wall. In some examples, the elongate member 204 may be substantially rigid, such that it maintains an approximately straight configuration during its insertion through the urethra. According to such examples, the distal portion of the elongate member 204 may be angled toward or away from various anatomical features surrounding the urethra, e.g., one or more lobes of the prostate gland, by adjusting the angular orientation of the proximal end of the elongate member 204 outside the body. The distal end of the elongate member 204 may comprise smooth, blunt, and/or beveled surfaces to avoid puncturing the urethral wall. Embodiments may include a sidewall opening 208 defined by a distal portion of the elongate member 204. The sidewall opening 208 may be sized and configured to accommodate passage of the hollow delivery needle therethrough.

Embodiments of the delivery device may include a variety of additional or substitute components and subassemblies, such as those described in U.S. Pat. No. 10,130,353, the entire contents of which are incorporated by reference herein.

Prior to insertion of any components of the delivery system, the implant recipient may undergo a regimen of antibiotics. Local anesthesia can be employed for the interventional procedure. A combination of an oral analgesic with a sedative or hypnotic component can be ingested by the subject. A topical anesthesia such as lidocaine liquids or gel can be applied to the bladder and urethra.

Examples of the implantation process may generally involve advancing the distal end of the tubular elongate member 204 containing a coaxial delivery needle distally through the urethra of a subject toward the urinary bladder until the distal end reaches the prostatic urethra, adjacent one or more lobes of the enlarged prostate gland targeted for compression. In some examples, the targeted lobe of the prostate gland may be chosen while the device extends through at least a portion of the prostatic urethra. In other embodiments, the targeted lobe is identified prior to the procedure, for example via ultrasound imaging. A distal portion of the elongate member 204 may, in some examples, be advanced into the bladder, where it may be positioned and/or rotated as needed to deploy the implant as desired within the targeted lobe upon retracting the elongate member 204 until its distal end returns to the prostatic urethra.

The distal end of the needle containing the distal anchor member 102 and elongate middle portion 104 may be advanced through and beyond the distal end of the elongate member 204, for example through the sidewall opening 208 defined near the distal end of the elongate member 204. The needle may curve and extend substantially orthogonally or perpendicularly away from the longitudinal axis of the elongate delivery member 204, for example in the manner shown and described in U.S. Pat. No. 11,298,115, the entire contents of which are incorporated by reference herein. Additional embodiments may involve advancing the needle through an opening defined by the distal end of the elongate member 204. Such embodiments may or may not define a sidewall opening. After exiting the elongate member 204, the clinician continues to advance the needle distally until its distal tip pierces through the urethral wall, targeted prostatic lobe, and outer prostatic capsule.

After the distal anchor member 102 contained within the delivery needle is positioned distally beyond the outer surface of the prostatic capsule, the needle may be retracted proximally toward the urethra, where the elongate member 204 remains, while the distal anchor member 102 and middle portion 104 are not retracted. As the needle is retracted, the distal anchor member 102 and middle portion 104 are unsheathed in a distal-to-proximal direction, such that the distal anchor member 102 of the implant is unsheathed first, outside the prostatic capsule. Free from the constraints of the inner lumen of the delivery needle, the distal anchor member 102 of the implant 100 may assume an unconstrained anchoring configuration, which may comprise an orientation transverse to the longitudinal axis of the needle and middle portion 104, thereby anchoring the implant 100 to the capsular side of the lobe.

Continued retraction of the needle unsheathes the middle portion 104 of the implant 100 within the lobe. The needle is further retracted until its distal tip is retracted through the urethral side of the lobe. Once the needle is fully retracted from the lobe and tension is applied to the middle portion 104 implanted therein, the expandable anchor member 106 of the implant 100 may be attached to the middle portion 104 at the urethral side of the lobe, where it expands against the outer surface of the lobe until the fully unconstrained anchoring configuration is attained.

In some embodiments, once the distal anchor member 102 and attached middle portion 104 have been deployed, with the needle retracted and middle portion 104 tensioned, the expandable anchor member 106 may be pushed by one or more components of the delivery device such that it captures the middle portion 104 transverse to the anchor axis. The middle portion 104 may then be cut just proximal to the expandable anchor member 106 to allow removal of the excess middle portion 104 not positioned within the prostatic lobe.

With the implant fully deployed, the needle and elongate member may be retracted proximally through the urethra until exiting the subject.

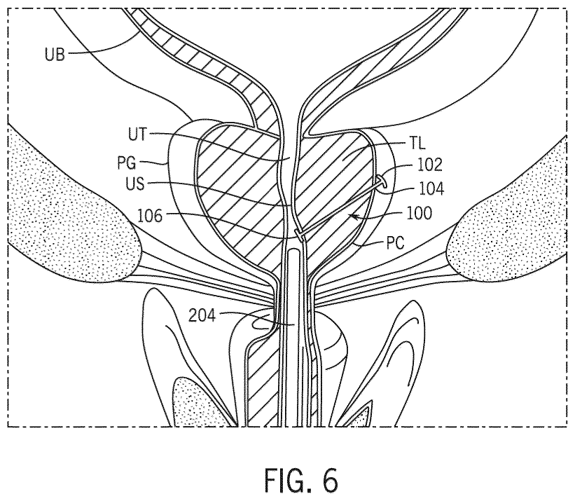

FIG. 6 is a cross-sectional view of an enlarged target lobe TL of a prostate gland PG and surrounding anatomy (urinary bladder UB, urethra UT) during implantation of a prostatic implant in a subject in accordance with embodiments disclosed herein. Implant 100 is depicted and referenced for case of illustration.

At the depicted stage of the implantation process, the distal anchor member 102 of the implant 100 has been positioned distally beyond the outer surface of the prostatic capsule PC of the target lobe TL, led by the distal end of a delivery needle, where the distal anchor member 102 is engaged with the prostatic capsule PC. The expandable anchor member 106 has been secured to the tensioned middle portion 104 and released from a tubular elongate member 204 on the urethral side US of the target lobe TL. The tubular elongate member 204 is in the process of retracting proximally back through the urethra.

The middle portion 104 has been extended through the target lobe TL in an approximately straight configuration. Upon tightening, the middle portion 104 pulls the distal anchor member 102 tightly against the surface of the prostatic capsule PC. Subsequent attachment of the expandable anchor member 106 to the middle portion 104 on the urethral side of the target lobe TL locks the tension in place. When the middle portion 104 is cut, the firmer capsule may spring back outward while the softer urethral side indents, thereby retracting the tissue of the target lobe TL and alleviating constriction of the adjacent prostatic urethra. After implantation, the expandable anchor member 106 provides an anchor having a footprint on the urethral side US of the target lobe TL defined by the area of its top (or bottom) surfaces in the unconstrained anchoring configuration, with the expandable portions 108a, 108b expanded outwardly relative to the longitudinal axis of the middle portion 104.

When deployed from the elongate member 204, the expandable anchor member 106 may expand in a direction substantially parallel to the urethral side US of the target lobe TL and in a direction substantially transverse to a longitudinal axis of the elongate member 204. The expandable anchor member may be secured to the middle portion 104 by movement in a direction generally parallel to the longitudinal axis of the elongate member 204, while expanding in a direction generally perpendicular to the longitudinal axis.

Depending in part on its material composition, the expandable anchor member 106 may substantially conform to the surface of the urethral side of the target lobe TL. The contact surface of the target lobe TL may not be uniformly flat or planar, however, such that one or more portions of the expandable anchor member 106 may not be perfectly or even substantially parallel to or flush with the surface of the lobe. The relative orientation between the expandable anchor member 106 and the lobe may thus depend at least in part on the surface features of the lobe.

The expandable anchor member 106 may have an advantageously large footprint relative to preexisting anchor devices and components, especially those lacking expandable portions. The large footprint increases the volume of prostatic tissue retracted after implantation, which may enhance the effectiveness of the implant as a whole by opening the constricted prostatic urethra to a greater extent than achieved via preexisting devices. At least in part because of the increase in tissue retraction, the implants described herein may exert compression forces sufficient to compress an enlarged prostate gland to its original size, i.e., a size that does not impinge on the prostatic urethra. The tension of the implant 100 may maintain a constant, permanent compression force on the targeted lobe TL. The implant 100 may not loosen over time, either via stretching and/or remodeling of the targeted lobe into a more relaxed, compressed state, thus enhancing the long-term therapeutic effect of the implant 100. As the targeted lobe contracts or is reduced over time, the implant 100 maintains its tension on the lobe, thus maintaining and potentially improving the therapeutic effect of the implant 100.

Implants having different configurations and properties may be used for different cases. For instance, expandable anchor members having larger surface areas may be selected to hold back larger amounts of prostatic tissue. Accordingly, methods of treating enlarged prostate glands may initially involve determining one or more properties of an enlarged prostate gland, such as its size and/or stiffness, and selecting an implant based on the properties. Other embodiments may feature a “one-size-fits-all” implant configured to accommodate any or most prostate glands.

In some cases, implant selection may also involve determining the number of implants to employ, with more implants typically required for larger, longer, and/or softer prostate glands. The large footprint of the expandable anchor portions disclosed herein may reduce the number of implants necessary per enlarged lobe, such that one of the disclosed implants may provide substantially the same or even greater tissue retraction and compression relative to two or more differently configured implants, including those having smaller proximal footprints.

Embodiments of the implant 100 may also feature one or more markers used to determine whether insertion of the implant 100 is complete. For instance, the distal anchor member 102 may include radiopaque marker visible only after reaching the outer surface of the prostatic capsule PC. According to such embodiments, detection of the marker indicates that the distal anchor member 102 has been sufficiently advanced beyond the prostatic capsule.

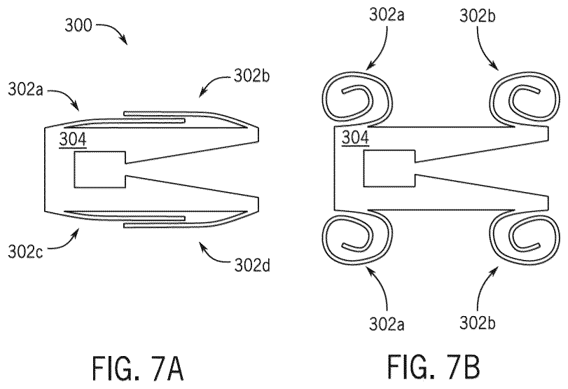

FIGS. 7A and 7B illustrate another embodiment of an expandable anchor member 300 of an implant used to compress at least a portion of a prostate gland in a subject. As shown, the expandable anchor member 300 may include four expandable portions 302a, 302b, 302c, 302d, each attached at one end to the body portion 304 of the expandable anchor member 300. In the constrained delivery configuration shown in FIG. 7A, the expandable portions 302a, 302b, 302c, 302d are compressed and substantially straightened near or against an outer wall of the body portion 304, such that the unattached ends of adjacent expandable portions may overlap. In the unconstrained anchoring configuration shown in FIG. 7B, the expandable portions 302a, 302b, 302c, 302d curl outwardly or laterally away from the body portion 304, forming spiral-like curved portions that individually and collectively expand the footprint of the expandable anchor portion 300.

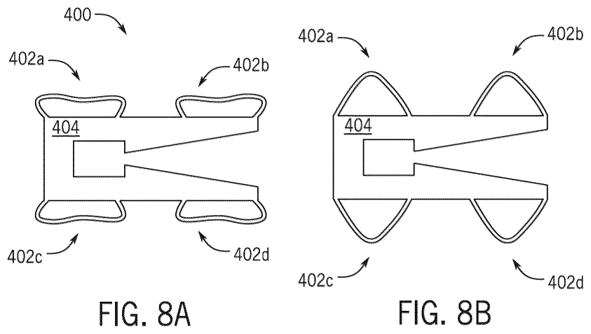

FIGS. 8A and 8B illustrate another embodiment of an expandable anchor member 400 of an implant used to compress at least a portion of a prostate gland in a subject. As shown, the expandable anchor member 400 may include multiple, e.g., four, expandable portions 402a, 402b, 402c, 402d, each attached at both ends to the body portion 404 of the expandable anchor member 400. In the constrained delivery configuration shown in FIG. 8A, the expandable portions 402a, 402b, 402c, 402d are compressed and substantially straightened near or against an outer wall of the body portion 404. In the unconstrained anchoring configuration shown in FIG. 8B, the expandable portions 402a, 402b, 402c, 402d expand outwardly or laterally away from the body portion 404, each forming arch-like curved portions or wings that individually and collectively expand the footprint of the expandable anchor portion 400.

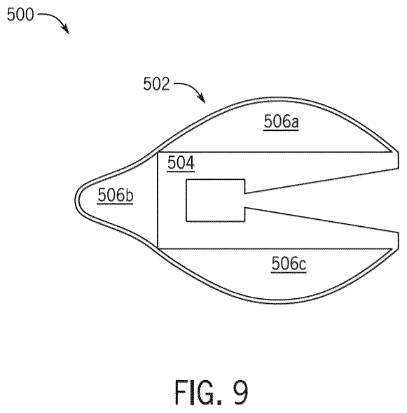

FIG. 9 illustrates another embodiment of an expandable anchor member 500 of an implant used to compress at least a portion of a prostate gland in a subject. As shown, the expandable anchor member 500 may include a single expandable portion 502 that extends around three sides of the body portion 504 of the implant 500, forming three expansion gaps 506a, 506b, 506c. Alternatively, the expandable portion 502 may comprise three distinct portions each attached at both ends to the body portion 504 of the expandable anchor member 500. The size of the expansion gaps may decrease in the constrained delivery configuration and increase in the unconstrained anchoring configuration.

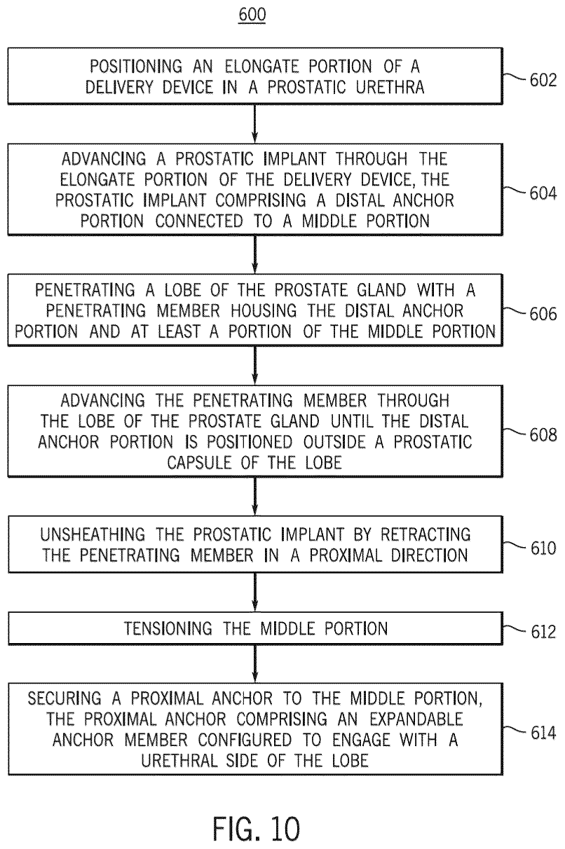

FIG. 10 is a flow diagram of a non-limiting example of a method 600 of delivering an implant to a prostate gland in accordance with embodiments of the present disclosure. The example method 600 may be performed to treat or alleviate symptoms of an enlarged prostate gland, which may be caused by a variety of conditions, including but not limited to benign prostatic hyperplasia and/or a variety of cancerous conditions that increase the size of the prostate gland, for example via tumor formation. The method may be implemented by one or more of the disclosed devices, systems, and/or components thereof. One or more additional steps may be included, and certain steps may also be omitted or performed in a different sequence than depicted.

In the embodiment shown, the method 600 begins at step 602 by “positioning an elongate portion of a delivery device in a prostatic urethra.” Step 604 of the method 600 may involve “advancing a prostatic implant through the elongate portion of the delivery device, the prostatic implant comprising a distal anchor portion connected to a middle portion.” In various embodiments, the middle portion may be or comprise a suture. Step 606 of the method 600 may involve “penetrating a lobe of the prostate gland with a penetrating member housing the distal anchor portion and at least a portion of the middle portion.” In various embodiments, the penetrating member may comprise a hollow delivery needle. Step 608 may involve “advancing the penetrating member through the lobe of the prostate gland until the distal anchor portion is positioned outside a prostatic capsule of the lobe.” Step 610 may involve “unsheathing the prostatic implant by retracting the penetrating member in a proximal direction.” Step 612 may involve “tensioning the middle portion.” Step 614 may involve “securing a proximal anchor to the middle portion, the proximal anchor comprising an expandable anchor member configured to engage with a urethral side of the lobe.” In various embodiments, the expandable anchor member, when deployed from a lumen of the elongate portion of the delivery device, may expand in a direction substantially parallel to the urethral side of the prostate gland and in a direction substantially transverse to a longitudinal axis of the delivery device. In various embodiments, the expandable anchor member may assume a compressed configuration within the lumen of the elongate delivery device and an expanded configuration outside the lumen of the elongate delivery device.

### Examples

The above Detailed Description includes references to the accompanying drawings, which form a part of the Detailed Description. The Detailed Description should be read with reference to the drawings. The drawings show, by way of illustration, specific embodiments in which the present accessory devices and associated methods can be practiced. These embodiments are also referred to herein as “examples.”

The Detailed Description is intended to be illustrative and not restrictive. For example, the above-described examples (or one or more features or components thereof) can be used in combination with each other. Other embodiments can be used, such as by one of ordinary skill in the art upon reviewing the above Detailed Description. Also, various features or components have been or can be grouped together to streamline the disclosure. This should not be interpreted as intending that an unclaimed disclosed feature is essential to any claim. Rather, inventive subject matter can lie in less than all features of a particular disclosed embodiment. Thus, the following claim examples are hereby incorporated into the Detailed Description, with each example standing on its own as a separate embodiment:

In Example 1, an implant device for treating benign prostatic hyperplasia by compressing a prostate gland includes a first anchor member configured to engage with an outer surface of a prostatic capsule of the prostate gland; a second, expandable anchor member configured to engage with a urethral side of the prostate gland; and a tensionable element that extends between the first anchor member and the second, expandable anchor member. The second, expandable anchor member, when deployed from a lumen of an elongate delivery device, expands in a direction substantially parallel to the urethral side of the prostate gland and in a direction substantially transverse to a longitudinal axis of the delivery device. The second, expandable anchor member assumes a compressed configuration within the lumen of the elongate delivery device and an expanded configuration outside the lumen of the elongate delivery device.

In Example 2, the second, expandable anchor member may include one or more expandable wings or curved portions.

In Example 3, the expandable wings or curved portions may comprise Nitinol or spring-temper steel.

In Example 4, a surface area of the second, expandable anchor member may be greater in the expanded configuration than in the compressed configuration.

In Example 5, the second, expandable anchor member may include a seating region that grips the tensionable element upon attachment of the expandable anchor member to the tensionable element.

In Example 6, the second, expandable anchor member may be configured for attachment to the tensionable element at the urethral side of the prostate gland.

In Example 7, the first anchor member may be configured to be constrained to a generally straight configuration aligned with a longitudinal axis of the elongate delivery device when positioned within the lumen.

In Example 8, the tensionable element comprises a suture portion.

In Example 9, the first anchor member may assume an unconstrained configuration upon deployment from the elongate delivery device.

In Example 10, the second, expandable anchor member may be biased to assume the expanded configuration.

In Example 11, an implant device for treating benign prostatic hyperplasia by compressing a prostate gland includes a first anchor member configured to engage with an outer surface of a prostatic capsule of the prostate gland; a second, expandable anchor member defining an internal slot and configured to engage with a urethral side of the prostate gland; and a tensionable element that extends between the first anchor member and the second, expandable anchor member. The second, expandable anchor member, when deployed from a lumen of an elongate delivery device, expands in a direction substantially parallel to the urethral side of the prostate gland and in a direction substantially transverse to a longitudinal axis of the delivery device. The second, expandable anchor member assumes a compressed configuration within the lumen of the elongate delivery device and an expanded configuration outside the lumen of the elongate delivery device. The internal slot of the second, expandable anchor member compresses the tensionable element.

In Example 12, the expandable anchor member may include compressive prongs configured to receive the tensionable element.

In Example 13, the second, expandable anchor member may include one or more expandable wings or curved portions.

In Example 14, a surface area of the second, expandable anchor member may be greater in the expanded configuration than in the compressed configuration.

In Example 15, the tensionable element comprises a suture portion.

In Example 16, a system for treating benign prostatic hyperplasia includes a delivery device comprising a handle assembly connected to an elongate portion, the elongate portion comprising a tubular member defining a first inner lumen and a second inner lumen; a hollow delivery needle having a sharp distal tip and configured to be extended through the first inner lumen of the tubular member upon activation of the handle assembly; and a prostatic implant configured to be extended through the tubular member. The prostatic implant includes a first anchor member configured to engage with an outer surface of a prostatic capsule of a prostate gland; a second, expandable anchor member configured to engage with a urethral side of the prostate gland; and a tensionable element that extends between the first anchor member and the second, expandable anchor member. The second, expandable anchor member, when deployed from the second inner lumen of the tubular member, expands in a direction substantially parallel to the urethral side of the prostate gland and in a direction substantially transverse to a longitudinal axis of the delivery device. The first anchor member of the prostatic implant is configured to be inserted through a lobe of the prostate gland, led by the sharp distal tip of the hollow delivery needle, until the first anchor member engages with the outer surface of the prostatic capsule upon retraction of the hollow delivery needle in a proximal direction into the lobe.

In Example 17, the second, expandable anchor member may assume a compressed configuration within the second inner lumen of the tubular member and an expanded configuration outside the second inner lumen of the tubular member.

In Example 18, the second, expandable anchor member may include one or more expandable wings or curved portions.

In Example 19, a surface area of the second, expandable anchor member may be greater in the expanded configuration than in the compressed configuration.

In Example 20, the second, expandable anchor member may include compressive prongs and a seating region configured to receive the tensionable element.

### Closing Notes

Certain terms are used throughout this patent document to refer to particular features or components. As one skilled in the art appreciates, different people may refer to the same feature or component by different names. This patent document does not intend to distinguish between components or features that differ in name but not in function.

For the following defined terms, certain definitions shall be applied unless a different definition is given elsewhere in this patent document. The terms “a,” “an,” and “the” are used to include one or more than one, independent of any other instances or usages of “at least one” or “one or more.” The term “or” is used to refer to a nonexclusive or, such that “A or B” includes “A but not B,” “B but not A,” and “A and B.” All numeric values are assumed to be modified by the term “about,” whether or not explicitly indicated. The term “about” generally refers to a range of numbers that one of skill in the art would consider functionally equivalent to the recited value (e.g., having the same function or result). In many instances, the term “about” can include numbers that are rounded to the nearest significant figure. The recitation of numerical ranges by endpoints includes all numbers and sub-ranges within and bounding that range (e.g., 1 to 4 includes 1, 1.5, 1.75, 2, 2.3, 2.6, 2.9, etc. and 1 to 1.5, 1 to 2, 1 to 3, 2 to 3.5, 2 to 4, 3 to 4, etc.). The terms “patient” and “subject” are intended to include mammals, such as for human or veterinary applications. The terms “distal” and “proximal” are used to refer to a position or direction relative to the treating clinician. “Distal” and “distally” refer to a position that is distant from, or in a direction away from, the treating clinician. “Proximal” and “proximally” refer to a position that is near, or in a direction toward, the treating clinician.

The scope of the invention should be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended; that is, a device, kit or method that includes features or components in addition to those listed after such a term in a claim are still deemed to fall within the scope of that claim. Moreover, in the following claims, the terms “first,” “second” and “third,” etc. are used merely as labels, and are not intended to impose numerical requirements on their objects.

The Abstract is provided to allow the reader to quickly ascertain the nature of the technical disclosure. It is submitted with the understanding that it will not be used to interpret or limit the scope or meaning of the claims.

What is claimed is:

1. An implant device for treating benign prostatic hyperplasia by compressing a prostate gland, comprising:
a first anchor member configured to engage with an outer surface of a prostatic capsule of the prostate gland;
a second, expandable anchor member configured to engage with a urethral side of the prostate gland; and
a tensionable element that extends between the first anchor member and the second, expandable anchor member,
wherein the second, expandable anchor member, when deployed from a lumen of an elongate delivery device, expands in a direction substantially parallel to the urethral side of the prostate gland and in a direction substantially transverse to a longitudinal axis of the delivery device, and
wherein the second, expandable anchor member assumes a compressed configuration within the lumen of the elongate delivery device and an expanded configuration outside the lumen of the elongate delivery device.

2. The implant device of claim 1, wherein the second, expandable anchor member comprises one or more expandable wings or curved portions.

3. The implant device of claim 2, wherein the expandable wings or curved portions comprise Nitinol or spring-temper steel.

4. The implant device of claim 1, wherein a surface area of the second, expandable anchor member is greater in the expanded configuration than in the compressed configuration.

5. The implant device of claim 1, wherein the second, expandable anchor member includes a seating region that grips the tensionable element upon attachment of the expandable anchor member to the tensionable element.

6. The implant device of claim 1, wherein the second, expandable anchor member is configured for attachment to the tensionable element at the urethral side of the prostate gland.

7. The implant device of claim 1, wherein the first anchor member is configured to be constrained to a generally straight configuration aligned with a longitudinal axis of the elongate delivery device when positioned within the lumen.

8. The implant device of claim 1, wherein the tensionable element comprises a suture portion.

9. The implant device of claim 1, wherein the first anchor member assumes an unconstrained configuration upon deployment from the elongate delivery device.

10. The implant device of claim 1, wherein the second, expandable anchor member is biased to assume the expanded configuration.

11. An implant device for treating benign prostatic hyperplasia by compressing a prostate gland, comprising:
a first anchor member configured to engage with an outer surface of a prostatic capsule of the prostate gland;
a second, expandable anchor member defining an internal slot and configured to engage with a urethral side of the prostate gland; and
a tensionable element that extends between the first anchor member and the second, expandable anchor member,
wherein the second, expandable anchor member, when deployed from a lumen of an elongate delivery device, expands in a direction substantially parallel to the urethral side of the prostate gland and in a direction substantially transverse to a longitudinal axis of the delivery device, and
wherein the second, expandable anchor member assumes a compressed configuration within the lumen of the elongate delivery device and an expanded configuration outside the lumen of the elongate delivery device,
wherein the internal slot of the second, expandable anchor member compresses the tensionable element.

12. The implant device of claim 11, wherein the expandable anchor member includes compressive prongs configured to receive the tensionable element.

13. The implant device of claim 11, wherein the second, expandable anchor member comprises one or more expandable wings or curved portions.

14. The implant device of claim 11, wherein a surface area of the second, expandable anchor member is greater in the expanded configuration than in the compressed configuration.

15. The implant device of claim 11, wherein the tensionable element comprises a suture portion.

16. A system for treating benign prostatic hyperplasia, comprising:
a delivery device comprising a handle assembly connected to an elongate portion, the elongate portion comprising a tubular member defining a first inner lumen and a second inner lumen;
a hollow delivery needle having a sharp distal tip and configured to be extended through the first inner lumen of the tubular member upon activation of the handle assembly; and
a prostatic implant configured to be extended through the tubular member, the prostatic implant comprising:
a first anchor member configured to engage with an outer surface of a prostatic capsule of a prostate gland;
a second, expandable anchor member configured to engage with a urethral side of the prostate gland; and
a tensionable element that extends between the first anchor member and the second, expandable anchor member,
wherein the second, expandable anchor member, when deployed from the second inner lumen of the tubular member, expands in a direction substantially parallel to the urethral side of the prostate gland and in a direction substantially transverse to a longitudinal axis of the delivery device, and
wherein the first anchor member of the prostatic implant is configured to be inserted through a lobe of the prostate gland, led by the sharp distal tip of the hollow delivery needle, until the first anchor member engages with the outer surface of the prostatic capsule upon retraction of the hollow delivery needle in a proximal direction into the lobe.

17. The system of claim 16, wherein the second, expandable anchor member assumes a compressed configuration within the second inner lumen of the tubular member and an expanded configuration outside the second inner lumen of the tubular member.

18. The system of claim 16, wherein the second, expandable anchor member comprises one or more expandable wings or curved portions.

19. The system of claim 16, wherein a surface area of the second, expandable anchor member is greater in the expanded configuration than in the compressed configuration.

20. The system of claim 16, wherein the second, expandable anchor member includes compressive prongs and a seating region configured to receive the tensionable element.
